(19) United States
(12) Patent Application Publication
(10) Pub. No.: US 2010/0070010 A1
(43) Pub. Date: Mar. 18, 2010

(54) Electrostimulation Device

(76) Inventor: Brian Arthur Simpson, Vale of Glamorgan (GB)

Correspondence Address: GrayRobinson, P. A., 401 E. LAS OLAS BLVD, SUITE 1850, FORT LAUDERDALE, FL, 33301, US

(21) Appl. No.: 12/224,758

(22) PCT Filed: Mar. 7, 2007
(86) PCT No.: PCT/GB2007/000791

§ 371 Date: Jul. 8, 2009

(30) Foreign Application Priority Data
Mar. 7, 2006 (GB) 0604613.0

(51) Int. Cl. A61N 1/05; A61N 1/04

(52) U.S. Cl. 607/117; 607/152; 607/148

(57) ABSTRACT

An electrostimulation device (e.g., 100) consists of a single paddle (e.g., 102) and at least one lead (e.g., 110) for connecting the electrostimulation device to a pulse generator. The paddle has at least one electrode (e.g., 106) for providing electrical stimulation. The electrode(s) is provided in an area (A) of the paddle and a ratio of the width of the area to the length of the area is greater than 0.5. The present invention also relates to a paddle (e.g., 102) implantable in a vertebra.

### FIELD OF THE INVENTION

This invention relates to an electrostimulation device, along with associated methods of use and manufacture. A particular, but not exclusive, application of the invention is electrical stimulation of the cervical spinal cord for relief of chronic intractable neuropathic and ischaemic pain.

### BACKGROUND TO THE INVENTION

Neuropathic pain begins or is caused by damage or dysfunction to the nervous system and is extremely difficult to manage. People are considered to have chronic pain if their symptoms persist for at least 6 months or if they have symptoms that last longer than expected for tissue healing or resolution of an underlying disease.

Although the actual number is unknown, a proportion of people with chronic neuropathic pain fail to obtain pain relief from pharmacological therapies despite adequate and reasonable efforts to use them. These people are said to have intractable neuropathic pain and they are the target population for Spinal Cord Stimulation (SCS).

Similar chronic pain can be due to inadequate blood supply (ischaemia). The above comments apply equally to such ischaemic pain.

Electrical stimulation of the spinal cord by means of electrodes implanted in the spine has been used since 1967 and has since been acknowledged to be an effective method for providing pain relief in some forms of chronic pain. SCS is a reversible pain therapy which involves the application of low-voltage electrical pulses directly to the spinal cord to manage chronic intractable neuropathic pain of the trunk and limbs.

Electrodes may be either positioned along a flexible elongate type electrostimulation device, which is inserted through a needle such as a standard 14 gauge Touhy needle, or on a substantially flexible flat paddle type electrostimulation device which requires an open operation for positioning. There is evidence to suggest that a paddle electrostimulation device functions better in certain respects than an elongate electrostimulation device e.g. electrodes on a paddle electrostimulation device face the spinal cord and therefore project the current directly to the cord, while electrodes on an elongate electrostimulation device are wrapped around the lead portion such that only a small proportion of the current is projected at the spinal cord. Furthermore, in some situations it is impossible to feed an elongate electrostimulation device through a needle and up to the required vertebral level due to epidural scar tissue, in this instance a paddle electrostimulation device is the only viable option.

A paddle electrostimulation device is implanted in the epidural space touching dura mater surrounding the spinal cord. However, placing the electrodes at the correct position along the spinal cord is a pre-requisite for satisfactory function. The commonest cause of failure to maintain pain relief is dislodgement of the electrode array of the electrostimulation device. Paddle electrostimulation devices are more stable than elongate electrostimulation devices but due to their long length, even they are prone to dislodgement when placed in the relatively mobile cervical spine. Short paddle length is a feature of a system designed by the applicant and manufactured by Advanced Neuromodulation Systems Inc, Plano, Tex., USA (Lamitrode 22™. This has proved to be considerably more stable than other systems (Simpson BA, Bassett G, Davies K, Herbert C, Pierri M. Cervical spinal cord stimulation for pain: a report on 41 patients. Neuromodulation 2003; 6: 20-26; and Simpson BA “A new dual bipolar electrode system for spinal cord stimulation” First International Congress of the International Neuromodulation Society, Rome, Italy. 1992 pp 13-17 Monduzzi Editore SpA, Bologna). However the Lamitrode 22™ comprises two separate paddles and requires a complex procedure for its insertion including a laminectomy (the removal of the back of one or more vertebrae).

There is therefore a need for a less invasive method of maintaining electrical stimulation using a paddle type electrostimulation device placed in the cervical spine.

### OBJECT OF THE INVENTION

It is an object of the present invention at least in preferred embodiments to provide a less invasive and more effective apparatus and method for electrical stimulation of the cervical spinal cord which overcomes the problem of dislodgement and thereby maintains pain relief.

### SUMMARY OF THE INVENTION

Viewed from a first aspect, the present invention relates to an electrostimulation device consisting of a single paddle and at least one lead for connecting to a pulse generator, a plurality of electrodes for providing electrical stimulation being provided in an area of the paddle; wherein the ratio of the width of said area to the length of said area is greater than 0.5. The length of the area is measured along a longitudinal axis of the paddle and the width of said area along a transverse axis. The at least one lead is typically connected to the bottom of the paddle and the longitudinal axis extends from the top of the paddle to the bottom of the paddle.

The area in which the electrodes are provided is preferably generally rectangular. In, preferred embodiments, the ratio of the width of the area to the length of the area is between 0.5 and 1.75 inclusive. Most preferably, the ratio is between 0.75 and 1.25 inclusive. The ratio may be approximately 0.75 or 1.

The applicant has recognised that it is particularly desirable that the paddle does not extend across more than one vertebra. The likelihood of the paddle being moved or dislodged after it has been implanted is thereby reduced. The area over which the electrodes are arranged preferably has a length of less than 25 mm. The length is preferably approximately 21 mm. More preferably, the area has a length of less than 20 mm. The length is preferably approximately 16 mm.

The area preferably has a width greater than 10 mm and less than 30 mm. More preferably, the area preferably has a width greater than 12 mm and less than 25 mm. The width is preferably approximately 16 mm.

Viewed from a further aspect, the present invention relates to an electrostimulation device consisting of a single paddle and at least one lead for connecting to a pulse generator, at least one electrode being provided on said paddle for providing electrical stimulation; wherein the paddle has a length of less than 25 mm. The paddle may have a length of less than or equal to 16 mm or 21 mm depending on its intended use. The paddle preferably comprises a plurality of electrodes. Thus, the present invention at least in preferred embodiments provides a short-paddle electrostimulation device having various configurations of electrodes positioned along its length and across its width.

The electrostimulation device is intended to be implanted in a human or animal body.

The paddle preferably has a width greater than 12 mm and less than 25 mm. More preferably the paddle has a width of between 14 mm and 18 mm.

The electrodes provided on the paddles described herein are preferably arranged in three or more columns. The electrodes are preferably arranged in three or more rows. The electrodes are preferably positioned on the paddle such that the various configurations facilitate optimal stimulation of various targets. The electrode configurations are compact allowing the paddle to remain sufficiently short, spanning no more than one vertebra, reducing the risk of dislodgement on movement. In contrast, existing commonly used paddle designs are two or more times the length and therefore cross “motion segments” increasing the risk of dislodgement on movement.

The electrodes may all be connected to each other by one or more internal wires. Alternatively, each electrode may be provided with a dedicated wire. In a further alternative, the electrodes in the array are provided in two or more groups and the electrodes in each group are connected by internal wires and a dedicated wire is provided for each group of electrodes. These latter arrangements enable the application of current to the electrodes to be controlled, for example to provide 2, 3, 4, 5, 6, or more channels.

The paddle preferably has between 8 and 16 electrodes in total. The electrodes may be oval and/or circular and preferably each have a width or diameter of between 0.5 mm and 5.0 mm and are preferably each between 2 mm and 5 mm long.

The at least one lead preferably extends from the bottom of the paddle. The at least one lead is preferably provided with a connector for connecting to a pulse generator. The least one lead may be connected to a pulse generator.

A preferred embodiment of the present invention provides a paddle which is implantable in a vertebra to provide electrical stimulation of a cervical spinal cord. The length of the paddle is preferably less than the height of the vertebra in which it is to be implanted.

The paddle is preferably provided with at least one suture hole to allow it to be secured in place. The at least one lead is preferably provided with one or more anchors.

Suture holes (not shown) may be positioned at various points around the outer edge of the paddle through which to secure the paddle to the dura to further prevent dislodgement.

A second end of the electrostimulation device preferably remains proximal the incision through which the paddle is implanted into the body. The second end of the electrostimulation device is intended for connection to a power source such as any industry standard fully implantable pulse generator (IPG). A connector may be provided at said second end of the electrostimulation device for connecting the electrostimulation device to the (PG.

The at least one lead is preferably not bifurcated. Thus, the at least one lead preferably extends from the paddle to the pulse generator without branching. The electrostimulation device preferably consists of either a single lead or two separate leads. A single lead may, for example, be used for an electrostimulation device having 12 or less electrodes; and two separate leads may, for example, be used for an electrostimulation device having 12 or more electrodes.

An anchor is preferably provided for securing the lead of the electrostimulation device to the fascia at the incision site. The anchor may be in the form of a bead which is threaded over the lead(s) toward the paddle. It will be appreciated that more than one anchor may be provided along the length of the lead for anchoring it in position.

The electrostimulation device may comprise a single electrode or a plurality of electrodes spaced apart along and/or across the paddle. Each electrode may be made of any suitable conductive material; preferably they are made of platinum iridium. The electrodes may be formed integrally with the paddle or they may be formed as a separate component and attached to the paddle.

The front face of the paddle is preferably substantially planar.

The at least one lead may be substantially parallel to or coincident with the front face of the paddle in the region where it joins the paddle. Alternatively, said at least one lead may be inclined at an angle α to the front face of the paddle. The angle α is typically greater than 180°. The angle α is preferably between 225° and 315° and, most preferably, is approximately 270°.

Viewed from a further aspect, the present invention relates to an electrostimulation device comprising a paddle and at least one lead for connecting to a pulse generator, at least one electrode for providing electrical stimulation being provided in an area of the paddle; the at least one electrode being provided on a front face of the paddle; wherein said at least one lead joins the paddle at an angle α relative to the front face of the paddle.

The angle α between said at least one lead and the front face of the paddle is preferably greater than 180°. The angle α is preferably between 225° and 315° and, most preferably, is approximately 270°.

The at least one lead is preferably joined to an edge of the paddle.

Viewed from a still further aspect, the present invention relates to a paddle implantable in a vertebra, the paddle having at least one electrode for providing electrical stimulation; wherein the length of the paddle is less than or equal to the height of the vertebra. The at least one electrode is preferably suitable for providing electrical stimulation of a cervical spinal cord.

Viewed from a yet further aspect, the present invention relates to a paddle for providing electrical stimulation, the paddle having a plurality of electrodes; wherein said electrodes are arranged in three or more columns. The columns preferably lie substantially parallel to a longitudinal axis of the paddle. The longitudinal axis extends from the top of the paddle to the bottom of the paddle.

The electrodes are preferably also arranged over three or more rows. The rows of electrodes preferably extend substantially perpendicular to said longitudinal axis.

At least one lead is preferably provided for connecting said electrodes to a current generator. The at least one lead is preferably connected to the bottom of the paddle.

Viewed from a yet further aspect, the present invention relates to a method of implanting a paddle in a vertebra to provide electrical stimulation of a cervical spinal cord, the method comprising the step of selecting a paddle having a length less than or equal to the height of the vertebra in which it is to be implanted.

The proposed design incorporates the advantage of the applicant's previous design, the Lamitrode 22™, in a single paddle which requires a much simpler operation for its insertion, including substantially less bone removal. Thus, at least in preferred embodiments the present invention offers a practical, less invasive method of electrical stimulation, particularly in but not limited to, the cervical spinal cord, than other previously suggested methods.

Preferred embodiments of the present invention will now be described by way of example only with reference to the accompanying Figures.

### BRIEF DESCRIPTION OF THE DRAWINGS

FIG. 1 shows the back of an electrostimulation device in accordance with a first embodiment of the present invention;

FIG. 2 shows the front of the electrostimulation device of FIG. 1;

FIG. 3 shows a perspective view of the electrostimulation device according to the first embodiment;

FIG. 4 shows a plan view of an electrostimulation device according to a second embodiment of the present invention;

FIG. 5A shows a perspective view of a paddle according to a third embodiment of the present invention;

FIG. 5B shows a side view of the paddle according to the third embodiment of the present invention; and

FIGS. 6 to 10 show alternative electrode configurations on an electrostimulation device.

### DETAILED DESCRIPTION OF THE PREFERRED EMBODIMENTS

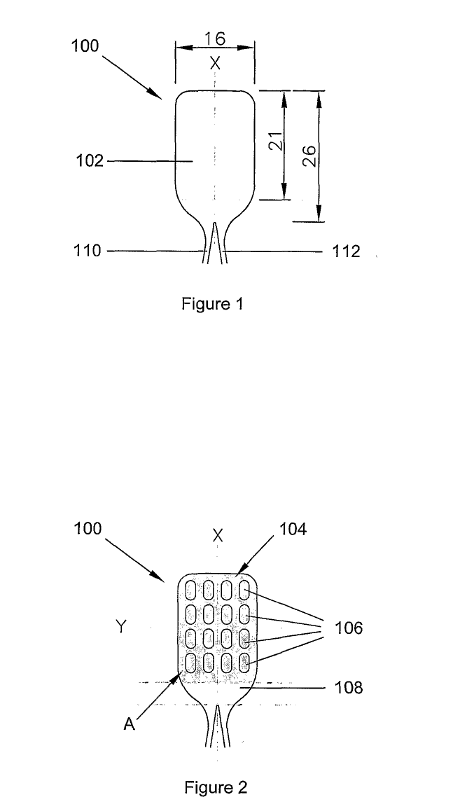

An electrostimulation device 100 in accordance with a first embodiment of the present invention is shown in FIGS. 1 and 2. The electrostimulation device 100 is implantable in a human or animal body to provide electrical stimulation of the cervical spinal cord.

The electrostimulation device 100 consists of a single paddle 102. An electrode array 104 of two or more electrodes 106 is provided in an area A (shaded in FIG. 2) on a front face 108 of the paddle 102. The electrode array 104 in the present embodiment is made up of sixteen electrodes 106 and, as shown in FIG. 2, the electrodes 106 are arranged in four rows and four columns. The columns of electrodes 106 extend substantially parallel to a longitudinal axis X of the paddle 102 and the rows extend substantially parallel to a transverse axis Y.

The electrodes 106 are oval and may be connected electrically to each other by a series of internal wires (not shown) in the paddle 102.

Preferably, however, each electrode 106 or one or more groups of electrodes 106 in the electrode array 104 are provided with dedicated internal wires to enable the application of current to different electrodes 106 in the electrode array 104 to be controlled, for example to allow control over 2, 3, 4, 5, 6 or more channels. The internal wires are connected to first and second leads 110, 112 to enable a current to be supplied to the electrode array 104. The first and second leads 110, 112 extend from the bottom of the paddle 102 and may comprise one or more wires. If the first and second leads 110, 112 are made up of a plurality of wires, the wires are typically sheathed with a suitable insulating material.

The first and second leads 110, 112 are connected to an implantable pulse generator (not shown). First and second connectors may optionally be provided on the free ends of the first and second leads 110, 112 (i.e. the ends remote from the paddle 102) to facilitate connection of the electrostimulation device 100 to the implantable pulse generator.

The electrodes 106 are made of platinum iridium and each have a width of approximately 2 mm and a length of approximately 4 mm. It will be appreciated that the size and shape of the electrodes 106 may be varied for different applications.

The paddle 102 has a total length of approximately 26 mm and a width of approximately 16 mm. The area A in which the electrode array 104 is provided has a width of approximately 16 mm and a length of approximately 21 mm extending from the distal end thereof. The paddle 102 has a neck portion provided between the area A and the first and second leads 110, 112. The corners of the paddle 108 are radiused.

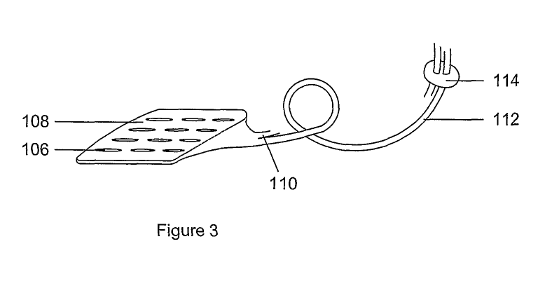

As shown in FIG. 3, the first and second leads 110, 112 are provided with an anchor 114 to help prevent migration of the electrostimulation device 100 after it has been implanted. The anchor 114 is a bead which is threaded over the first and second leads 110, 112 and fixed in position. In addition to the anchor 114 (or in place thereof), the paddle 108 may be provided with one or more suture holes (not shown) to allow the paddle 102 to be secured in position. The suture holes would typically be provided around the outer edge of the paddle 102.

The electrostimulation device 100 is implantable to provide electrical stimulation of the spinal cord. Since the electrostimulation device 100 is not bifurcated, it can be implanted with relative ease. Its relatively compact design allows the paddle 102 to be implanted with the removal of less bone. Furthermore, the length of the paddle 102 is such that, once implanted, it spans only a single vertebra and the risk of it being dislodged after it has been implanted is reduced.

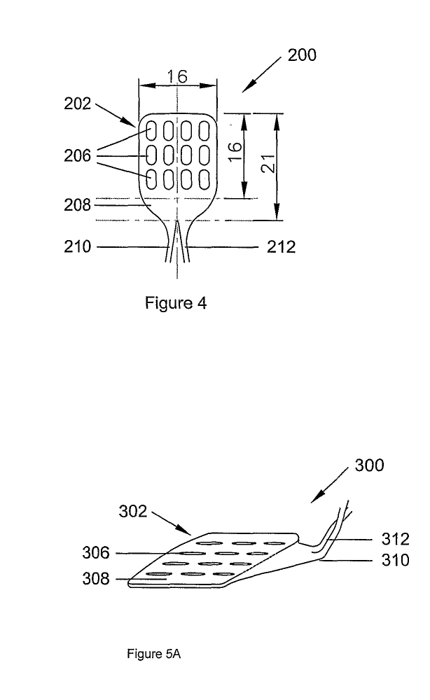

An electrostimulation device 200 according to a second embodiment of the present invention is shown in FIG. 4.

The electrostimulation device 200 consists of a single paddle 202 which is shorter than the paddle 102 of the first embodiment. Specifically, the paddle 202 has a length of approximately 16 mm. To accommodate the reduction in size of the paddle 202, the electrode array 204 consists of twelve electrodes 206 arranged in three rows and four columns. The electrodes 206 are each substantially the same size as those in the first embodiment.

The reduced length of the electrostimulation device 200 is advantageous since the paddle 202 may be implanted more readily. The likelihood of the paddle 202 being dislodged once it has been implanted is also reduced further.

A third embodiment of an electrostimulation device 300 in accordance with the present invention is shown in FIGS. 5A and 5B.

The paddle 302 is made of a transparent material and the electrodes 306 are visible through the back of the paddle 302, as shown in FIG. 5A.

In the first embodiment of the present invention the first and second leads 110, 112 extend away from the paddle 102 in a reference plane substantially coincident with the front face 108 of the paddle 102.

However, in the third embodiment, the first and second leads 310, 312 are predisposed to join the paddle 302 at an angle α relative to a reference plane substantially coincident with the front face 308 of the paddle 302. In the present embodiment the angle α is approximately 270°, but it may be between 225° and 315°. Thus, the first and second leads 310, 312 are predisposed to extend away from the back of the paddle, as shown in FIG. 5B.

The paddle 302 has a cover which biases the first and second leads 310, 312 in the desired direction. This arrangement provides two different ways in which the paddle 302 may be implanted. In certain applications this configuration can help to locate the paddle 302 when it is implanted and reduce the tendency of the paddle 302 to be dislodged after it has been implanted.

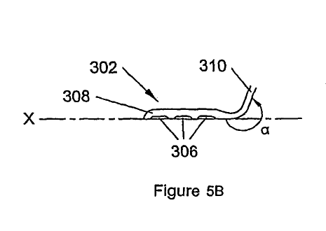

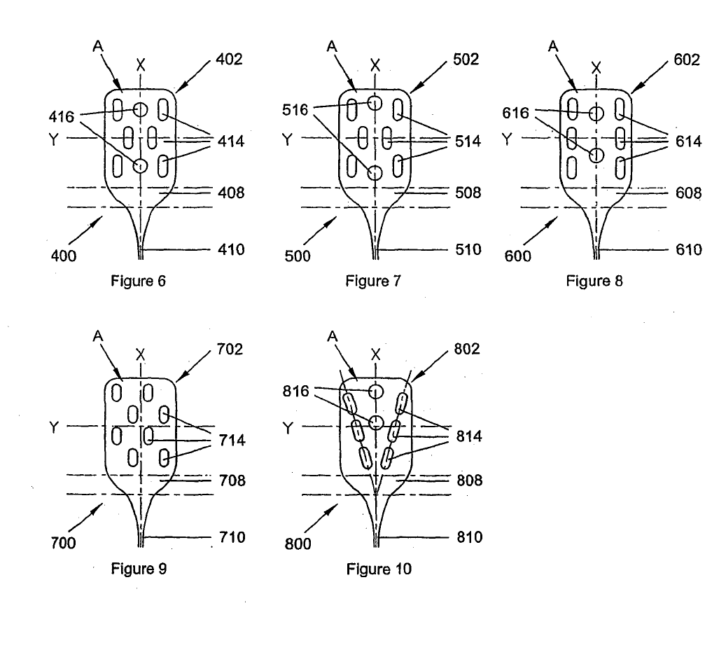

A fourth embodiment of an electrostimulation device 400 in accordance with the present invention is shown in FIG. 6.

The electrostimulation device 400 consists of a single lead 410 and a different arrangement of electrodes 406. The electrodes 406 are a combination of oval electrodes 414 and circular electrodes 416 arranged in three rows. The top and bottom rows of electrodes 406 consist of two oval electrodes 414 on each side of a circular electrode 416. The central row of electrodes 406 consists of two oval electrodes 414 located inwardly of the oval electrodes 414 in the top and bottom rows. The circular electrodes 416 are located along the longitudinal axis X of the paddle 402; and the oval electrodes 414 in the middle row are located along the transverse axis Y of the paddle 402.

A fifth embodiment of an electrostimulation device 500 in accordance with the present invention is shown in FIG. 7.

The fifth embodiment is similar to the fourth embodiment but the circular electrodes 516 are moved apart from each other along the longitudinal axis X of the paddle 502.

A sixth embodiment of an electrostimulation device 600 in accordance with the present invention is shown in FIG. 8.

The sixth embodiment is similar to the fourth embodiment but the oval electrodes 614 in the middle row are moved apart from each other along the transverse axis Y of the paddle 602. The oval electrodes 614 are thereby arranged in two columns of three.

A seventh embodiment of an electrostimulation device 700 in accordance with the present invention is shown in FIG. 9.

The seventh embodiment consists of only oval electrodes 714 arranged in four columns with two oval electrodes 714 in each column. The oval electrodes 714 in adjacent columns are offset from each other.

An eighth embodiment of an electrostimulation device 800 in accordance with the present invention is shown in FIG. 10.

The eighth embodiment consists of two columns of oval electrodes 814 and a pair of circular electrodes 816. Rather than being parallel, the columns of oval electrodes 814 define an acute internal angle such that the oval electrodes 814 proximal the single lead 810 are closer together than those distal from the lead 810. The circular electrodes 816 are arranged along the longitudinal axis X of the paddle 802.

The references herein to the top, bottom and sides of the paddle are for reference purposes only and are not intended to be limiting on the scope of the invention.

The described embodiments of the invention are only examples of how the invention may be implemented. Modifications, variations and changes to the described embodiments will occur to those having appropriate skills and knowledge. These modifications, variations and changes may be made without departure from the spirit and scope of the invention defined in the claims and its equivalents.

For example, it will be appreciated that any number of electrodes in any number of configurations may be provided along the length and across the width of a paddle with an overall dimension of 16 mm wide and between 21 mm and 26 mm long.

What is claimed is:

1. An electrostimulation device consisting of:
a single paddle;
at least one lead for connecting to a pulse generator; and
at least one electrode for providing electrical stimulation in an area of the paddle;
wherein the ratio of the width of said area to the length of said area is between 0.75 and 1.25 inclusive.

2. An electrostimulation device as claimed in claim 1, wherein said area has a length of less than 25 mm.

3. An electrostimulation device as claimed in claim 1, wherein said area has a width of greater than 10 mm.

4. An electrostimulation device consisting of:
a single paddle;
at least one lead for connecting to a pulse generator; and
at least one electrode being provided on said paddle for providing electrical stimulation;
wherein the paddle has a length of less than 25 mm.

5. An electrostimulation device as claimed in claim 1; wherein the at least one electrode comprises a plurality of electrodes.

6. An electrostimulation device as claimed in claim 5, wherein the electrodes are arranged in three or more columns.

7. An electrostimulation device as claimed in claim 6, wherein the electrodes are arranged in three or more rows.

8. An electrostimulation device as claimed in claim 5, wherein the electrodes are at least one of oval and circular.

9. An electrostimulation device as claimed in claim 1, wherein said at least one lead extends from the bottom of the paddle.

10. An electrostimulation device as claimed in claim 1, wherein said at least one lead is provided with a connector for connecting to a pulse generator.

11. An electrostimulation device as claimed in claim 1, wherein said at least one lead is connected to a pulse generator.

12. An electrostimulation device as claimed in claim 1, wherein said paddle is implantable in a vertebra to provide electrical stimulation of a cervical spinal cord.

13-14. (canceled)

15. An electrostimulation device as claimed in claim 1, wherein the at least one lead is connected to said paddle.

16. An electrostimulation device as claimed in claim 1, wherein an angle of greater than 180° is defined between said at least one lead and a front face of the paddle.

17. An electrostimulation device as claimed in claim 16, wherein an angle of approximately 270° is defined between said at least one lead and the front face of the paddle.

18-19. (canceled)

20. A paddle implantable in a vertebra, the paddle comprising:
at least one electrode for providing electrical stimulation; wherein the length of the paddle is less than or equal to the height of the vertebra.

21. A paddle as claimed in claim 20, wherein said at least one electrode is suitable for providing electrical stimulation of a cervical spinal cord.

22. A paddle for providing electrical stimulation, the paddle comprising:
a plurality of electrodes; wherein said electrodes are arranged in three or more columns.

23. A paddle as claimed in claim 22, wherein the electrodes are arranged in three or more rows.

24. A paddle as claimed in claim 20, wherein the at least one electrode comprises a plurality of electrodes and wherein the electrodes are arranged in at least three rows and at least three Columns.
